(19) United States
(12) Patent Application Publication
(10) Pub. No.: US 2020/0243180 A1
(43) Pub. Date: Jul. 30, 2020

(54) OPTIMIZED MEDICATION RECOMMENDATION USING A NEURAL NETWORK

(71) Applicant: International Business Machines Corporation, Armonk, NY (US)

(72) Inventors: John Mbari Wamburu, Nairobi (KE); Charles Muchiri Wachira, Nairobi (KE); Aisha Walcott, Nairobi (KE); Samuel Osebe, Nairobi (KE); Sekou Lionel Remy, Nairobi (KE); Katherine Anne Lyon Tryon, Hardy (KE)

(21) Appl. No.: 16/257,779

(22) Filed: Jan. 25, 2019

(51) Int. Cl. G16H 20/10; G16H 50/20; G16H 50/70; G16H 70/40; G16H 70/20

(52) U.S. Cl. CPC G16H 20/10; G16H 50/20; G16H 70/20; G16H 70/40; G16H 50/70

(57) ABSTRACT

A method includes: receiving a diagnosis of a medical condition; retrieving encoded guidelines for pharmaceutical treatment corresponding to the diagnosis; in response to the encoded guidelines, generating a list of recommended medications with related information; modifying the list of recommended medications, in response to personal context data obtained from at least one of a wearable device, a smart pill bottle, a patient health wallet, and an electronic health record; further modifying the list of recommended medications in response to environmental context data; generating an ordered list of recommended medications by yet further modifying the list of recommended medications in response to a patient similarity analysis; providing to a prescriber the ordered list of recommended medications; developing a logistical plan including a mode of delivery for one of the recommended medications; and programming an autonomous device to implement the mode of delivery.

### BACKGROUND

The present invention relates to the medical arts and to the electrical, electronic, and computer science arts, and more specifically, to processes for medication recommendation (prescribing).

Prescribing medication is both a science and an art. The science of prescribing involves identifying biochemical factors that contribute to a diagnosed disease process, and identifying medications that will alter the identified biochemical factors to mitigate the disease process. The art of prescribing involves considering various patient and environmental factors that will affect the efficacy of various medications if one of those formulations is prescribed to a particular patient, then selecting a medication to recommend in light of the biochemical, patient, and environmental factors.

### SUMMARY

Principles of the invention provide techniques for optimized medication recommendation using a neural network. In one aspect, an exemplary method includes receiving, by a guideline recommender engine, a diagnosis of a medical condition for a given patient; retrieving, by the guideline recommender engine, encoded guidelines for pharmaceutical treatment corresponding to the diagnosis; and in response to the encoded guidelines, generating, by a medication information engine, a list of recommended medications with related information. The exemplary method further includes modifying the list of recommended medications, by a personalized and contextualized medication recommender engine, in response to personal context data for the given patient that is obtained from at least one of a wearable device, a smart pill bottle, a patient health wallet, and an electronic health record; further modifying the list of recommended medications by the medication recommender engine in response to environmental context data for the given patient; and generating an ordered list of recommended medications by yet further modifying the list of recommended medications by the medication recommender engine in response to a patient similarity analysis that compares the personal context data of the given patient to personal context data of at least one other patient. Finally, the exemplary method includes providing to a prescriber by the medication recommender engine the ordered list of recommended medications.

In another aspect, an exemplary method includes receiving by a guideline recommender engine a diagnosis of a medical condition for a given patient; retrieving by the guideline recommender engine encoded guidelines for pharmaceutical treatment corresponding to the diagnosis; and in response to the encoded guidelines, generating by a medication information engine a list of recommended medications with related information. The exemplary method further includes modifying the list of recommended medications by a personalized and contextualized medication recommender engine in response to personal context data for the given patient that includes the given patient's personal preference for allowable side effects of medications.

In another aspect, an exemplary method includes receiving by a guideline recommender engine a diagnosis of a medical condition for a given patient; retrieving by the guideline recommender engine encoded guidelines for pharmaceutical treatment corresponding to the diagnosis; and in response to the encoded guidelines, generating by a medication information engine a list of recommended medications with related information. The exemplary method also includes modifying the list of recommended medications by a personalized and contextualized medication recommender engine in response to personal context data for the given patient that includes the given patient's financial income as well as financial cost for each medication on the list of recommended medications.

As used herein, “facilitating” an action includes performing the action, making the action easier, helping to carry the action out, or causing the action to be performed. Thus, by way of example and not limitation, instructions executing on one processor might facilitate an action carried out by instructions executing on a remote processor, by sending appropriate data or commands to cause or aid the action to be performed. For the avoidance of doubt, where an actor facilitates an action by other than performing the action, the action is nevertheless performed by some entity or combination of entities.

One or more embodiments of the invention or elements thereof can be implemented in the form of a computer program product including a computer readable storage medium with computer usable program code for performing the method steps indicated. Furthermore, one or more embodiments of the invention or elements thereof can be implemented in the form of a system (or apparatus) including a memory, and at least one processor that is coupled to the memory and operative to perform exemplary method steps. Yet further, in another aspect, one or more embodiments of the invention or elements thereof can be implemented in the form of means for carrying out one or more of the method steps described herein; the means can include (i) hardware module(s), (ii) software module(s) stored in a tangible computer readable storage medium (or multiple such media) and implemented on a hardware processor, or (iii) a combination of (i) and (ii); any of (i)-(iii) implement the specific techniques set forth herein.

In view of the foregoing, techniques of the present invention can provide substantial beneficial technical effects. For example, one or more embodiments provide one or more of:

A model constituting a rich set of attributes, including factors of environmental, financial, and physiological nature such as a given patient's side-effects tolerance, accessibility, and availability of drugs.

A cognitive approach by which a patient's context provides the model for the patient's environment; this is used to rank discrete practicality/convenience of medications for an individual rather than a statistical assessment for a population.

Use of prescribing guidelines and a patient's condition/diagnosis to determine a set of recommended medications.

A model that considers financial cost and timing of medication acquisition, as well as non-financial costs that a patient might consider barriers to adherence.

A model that focuses on a patient's ability to adhere to a medication treatment plan.

A method for configuring one or more autonomous devices (drone, self-driving vehicle, robotic home assistant) to implement logistics for obtaining medication.

These and other features and advantages of the present invention will become apparent from the following detailed description of illustrative embodiments thereof, which is to be read in connection with the accompanying drawings.

### BRIEF DESCRIPTION OF THE DRAWINGS

FIG. 1 depicts a cloud computing environment according to an embodiment of the present invention;

FIG. 2 depicts abstraction model layers according to an embodiment of the present invention;

FIG. 3 depicts a conceptual framework for optimized medication recommendation, according to an exemplary embodiment;

FIG. 4 depicts a logistical planner for medication access, according to an exemplary embodiment;

FIG. 5 depicts a tactical planner for medication delivery, according to an exemplary embodiment; and

FIG. 6 depicts a computer system that may be useful in implementing one or more aspects and/or elements of the invention, also representative of a cloud computing node according to an embodiment of the present invention.

### DETAILED DESCRIPTION

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

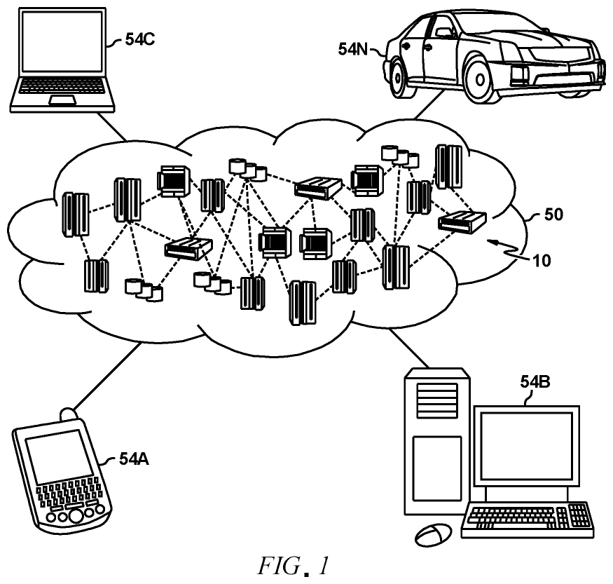

Referring now to FIG. 1, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 1 are intended to be illustrative only and that computing nodes 10 and cloud computing environment 50 can communicate with any type of computerized device over any type of network and/or network addressable connection (e.g., using a web browser).

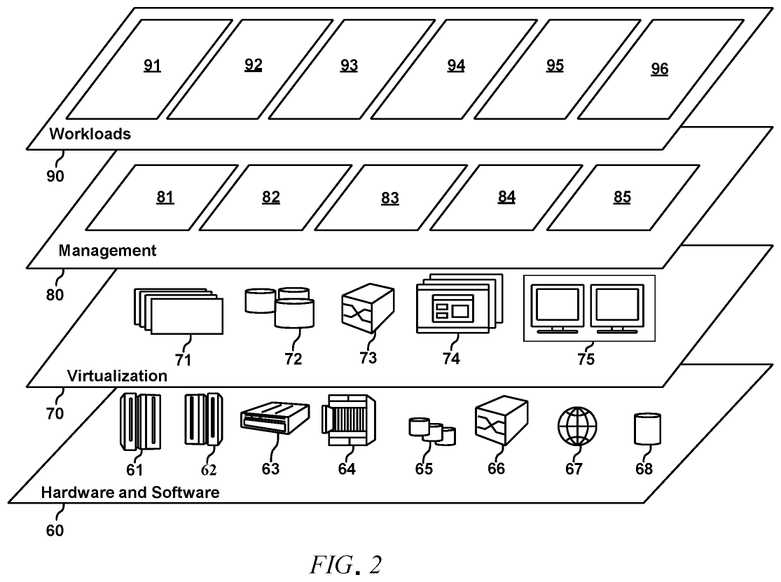

Referring now to FIG. 2, a set of functional abstraction layers provided by cloud computing environment 50 (FIG. 1) is shown. It should be understood in advance that the components, layers, and functions shown in FIG. 2 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and a medication recommendation system 96 that implements a method for generating an ordered list of recommended medications.

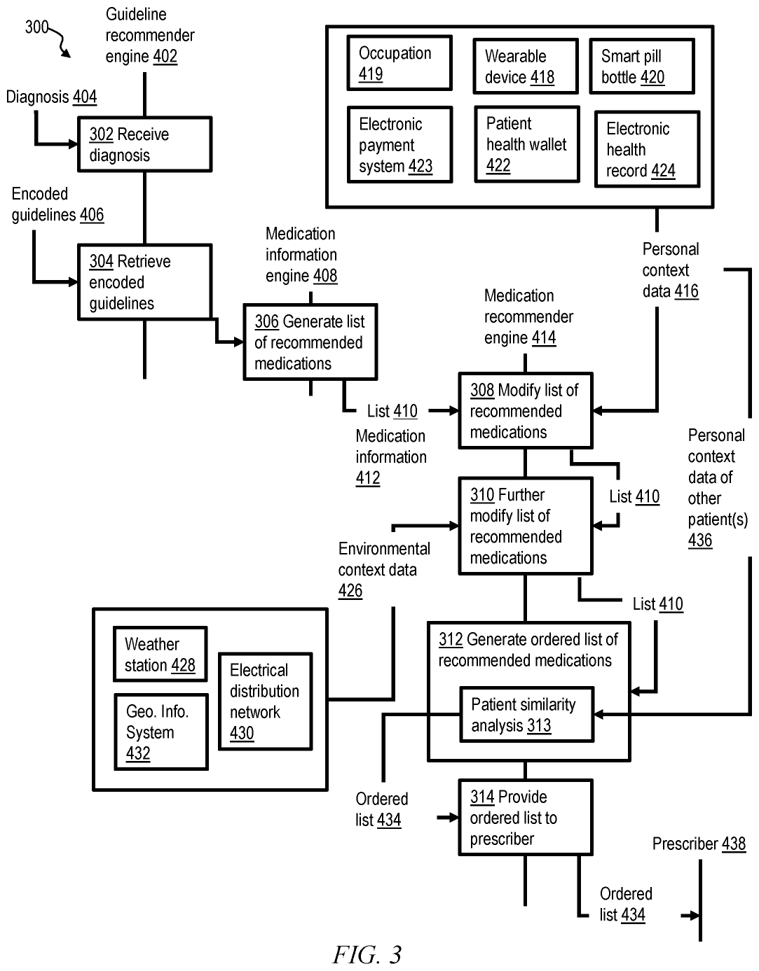

Referring to FIG. 3, in one or more embodiments, the method 300 that is implemented by the medication recommendation system generally includes, at 302, receiving by a guideline recommender engine 402 a diagnosis 404 of a medical condition for a given patient. Then, at 304, the method 300 continues to the step of retrieving, by the guideline recommender engine 402, encoded guidelines 406 for pharmaceutical treatment corresponding to the diagnosis 404. At 306, in response to the encoded guidelines 406, the method 300 includes generating by a medication information engine 408 a list 410 of recommended medications with related medication information 412.

At 308, the method 300 further includes modifying the list 410 of recommended medications by a personalized and contextualized medication recommender engine 414 in response to personal context data 416 for the given patient that is obtained from at least one of a wearable device 418, a smart pill bottle 420, a patient health wallet 422, and an electronic health record 424. Of course, appropriate privacy protections should be utilized when obtaining/handling/storing any personal data or information, such as obtaining informed consent, utilizing anonymization, utilizing encryption, and the like.

For the avoidance of confusion, a patient health wallet 422 is a system/application that is owned by the patient and allows the patient to access his or her health data and obtain an audit trail of the care providers who see or edit her or his data. An electronic health record 424 usually faces the care provider and the patient health wallet reads its data from the electronic health record. In addition a health wallet allows the patient to share his or her data with other care providers in other facilities. Also the patient health wallet allows a patient to communicate with care providers through an embedded messaging platform. Thus, the patient health wallet as explained above, has personal information which can be used to define the patient context that is relevant for a medication recommender. By carrying out natural language processing (NLP) on conversations module data of the patient health wallet, patient preferences can be deduced from the questions the patient asks care providers or the system, and the answers offered to same.

Meanwhile, a smart pill bottle 420 uses sensors for monitoring the status of the pill container, and a wireless transmitter to transmit this data to a server. One or more embodiments read this data and determine the level of adherence to a medication regimen from, say, missing pills. If certain non-adherence is detected the system invokes the conversations module of the patient health wallet, and, using targeted questions, tries to deduce the reason why the patient failed to adhere. Some of the reasons may be related to patients experiencing interactions on ingesting the drugs, patients not liking the size of the pills, etc. This information defines the patient context from a pill bottle perspective.

The personal context data 416 includes, for example, comparison of medication cost to patient income; comparison of potential adverse medication reactions to patient preferences; and comparison of potential administration modes to patient preferences. In one or more embodiments, the personal context data 416 also incorporates information about at least one of an electronic payment system 423 and the given patient's occupation 419.

In comparing medication cost to patient income, the medication recommender engine 414 considers total treatment cost T_{c} as the set {T_{c,1}, T_{c,2}, T_{c,3} . . . T_{c,n}} where each T_{c} is the cost of a given treatment plan, n is a number of possible treatment plans for the given patient, and T_{c,i}=dₚ*dₙᵤₘ*t_{d} with dₚ=Dosage price, dₙᵤₘ=Number of doses per day, and t_{d}=Time(days) for taking the medication under the given treatment plan. Then the medication recommender engine 414 calculates a medication cost factor for each recommended medication, d_{c}=f(norm, inc), where norm is the normalized cost of each recommended medication and inc is the income level of the patient, informed by occupation's annual household income obtained from, e.g., the electronic payment system 423 or the patient health wallet 422. The drug cost factor for each recommended medication is one of the inputs in the overall scoring function that provides a rank of the recommended meds.

In comparing adverse medication reactions to patient preferences, the medication recommender engine 414 considers a set of side effects K={S_{E1}, S_{E2}, S_{E3}, . . . , S_{En}}, a set of medication-to-medication interactions DI={di₁, di₂, di₃, . . . diₙ}, and a set of food interactions FI={fi₁, fi₂, fi₃, . . . fiₙ}, and computes for each recommended medication an adverse medication reaction(ADR) factor adr=f(cp, sev, po), where cp indicates client tolerance against a certain ADR based on his or her occupation; sev indicates severity of an ADR; and po indicates probability of occurrence of an ADR. Please note that “n” is intended as a general integer and is not necessarily the same for K, DI, FI . . . K is a set of all side effects associated with a given recommended medication. DI is a set of drug-drug interactions that a recommended drug will have with any active meds that a patient is currently taking. If a patient is being prescribed only one drug today and is not taking any other drug at home, this set will be empty. FI is a set of food interactions which a recommended drug will have with the patient diet. Each of these items in the 3 sets is called an adverse drug reaction (ADR). For each of the ADR (=K+DI+FI), the system will find the sev—severity and po—probability of occurrence which are usually available in Drug Information Services such as Drug Bank. The cp—client tolerance to an ADR is deduced from the patient's health wallet conversations data (e.g. a patient might have said in a text how headaches affect her or his life as opposed to one time vomiting or truck driver cannot tolerate medication that makes him or her drowsy). A function weighs the cp, sev and po for each ADR and an average of this function's output for all ADRs for a recommended drug is what is being called ADR factor—adr. Similar to the drug cost factor, above, the adr for each recommended medication is one of the inputs to the overall scoring function that provides a rank of the recommended meds.

Active medication data can either be obtained from the electronic health records 424 and/or the patient health wallet 422. Note that a patient's health wallet 422 might have information that is not on the EHR 424, e.g., a patient can record in the health wallet his or her purchase of over the counter medication.

Patient diet can be deduced from diet information entered in the patient health wallet 422, and also from internet-of-things (IoT) sensors. Tolerance to an ADR, and occupation, can be obtained by analyzing conversations about ADR that are stored in the patient health wallet 422. Information on adherence can be collected using smart pill bottles 420, as earlier explained. In comparing potential administration modes to patient preferences, the medication recommender engine 414 determines a recommended medication administration mode, da=f (df, cpm, freq) where df is a medication form, cpm is a client preference for mode of administration, and freq is a client preference for frequency of administration for each medication form.

Thus, the patient health wallet 422 and the EHR 424 are used to ensure that the correct data is used for decision-making, and that the communication between patient and providers is efficient, targeted, and auditable. The smart pill bottles 420 (i.e. a connected device) provide mechanisms to ensure that the patient receives reminders, and to capture temporally relevant information to help the patient avoid ADRs.

In considering patient context data 416, the medication recommender engine 414 establishes normalized variables that characterize each medication in the list 410. The variables include:

k=0: ADR free medication
k=1: High ADR factor
c=0: cheapest drug
c=1: most expensive drug
a=0: drug unavailable
a=1: drug available
f=0: least drug forms
f=1: most drug forms

Then, for each of the above variables, a weight W is assigned (Wₖ, W_{c}, Wₐ, and W_{f}, respectively), and an overall optimization score m is calculated for each of the medications in the encoded guidelines 406 according to

$$m = \frac{{W}_{k} K}{1 + {W}_{k} K} \times \frac{{W}_{c} C}{1 + {W}_{c} C} \times \frac{{W}_{a} A}{1 + {W}_{a} A} \times \frac{{W}_{f} F}{1 + {W}_{f} F} .$$

At 310, the method 300 includes further modifying the list 410 of recommended medications by the medication recommender engine 414 in response to environmental context data 426 for the given patient (e.g., accessibility or availability of a medication) that is obtained from at least one of a weather station 428, an electrical distribution network 430, and a geographical information system 432. This point about accessibility and availability relates to whether a recommended medication for prescribing is available and accessible. In some cases the patient may be prescribed medication that is not available in the current hospital's pharmacy For example, if power to the hospital has been interrupted, then refrigerated medications may be unavailable. In such circumstances, the patient would have to obtain the medications elsewhere. Such a situation begs the question, is the elsewhere accessible? Things which affect accessibility are, for example, weather which can ruin roads. The weather data is publicly accessible.

At step 310, the medication recommender engine 414 also considers medication availability in inventory at various dispensary/pharmacy locations ordered by distance from the given patient. The medication recommender engine 414 adjusts the distance from the given patient according to environmental context (e.g., weather, mode of transport) and calculates an accessibility function: acc=f(av, dist, ctx), where av: availability factor for each recommended medication—whether in a dispensary's inventory or not; dist: distance to the next available pharmacy or other dispensary; ctx: contextual factors that affect access (e.g., weather, mode of transport—walking, road vehicle, etc.). The medication recommender engine 414 further modifies the list 410 by ranking lower those medications for which the accessibility function acc falls outside an acceptable range of values, due for example to the medication not being available at any dispensary within a four hour distance from the given patient. Other acceptable ranges of values for the accessibility function may be selected by the given patient or by a prescriber, according to contextual factors such as population density, the given patient's available time and ability to travel for obtaining medication, etc.

At 312, the method 300 includes generating an ordered list 434 of recommended medications by yet further modifying the list 410 of recommended medications by the medication recommender engine 414 in response to a patient similarity analysis 313 that compares the personal context data 416 of the given patient to personal context data 436 of at least one other patient.

In one or more embodiments, the personal context data 436 for each of the other patients is obtained from each of the other patients' electronic health record (EHR), wearable device, smart pill bottle, or patient health wallet. For example, in one or more embodiments, the patient similarity analysis 313 compares the given patient to other patients, each of whom has taken a different medication from the list 410 of recommended medications. The patient similarity analysis 313 identifies a group of the other patients who are most similar to the given patient, and compares outcomes for the group of the other patients according to which medication from the list 410 each of the group of the other patients was prescribed. By ranking the “best” outcomes among the group of the other patients, the patient similarity analysis 313 produces the ordered list 434 of recommended medications. For example, in case the given patient has an acute (temporary, short-term) condition, the “best” outcomes will be those with the shortest time to recovery from the condition. On the other hand, in case the given patient has a chronic (permanent or long-term) condition, the “best” outcomes will be those with the fewest documented adverse medication reactions and the longest times at a stable dose of the prescribed medication. In one or more embodiments, the highest ranked medication will be the medication that scores well in both aspects.

At 314, the method 300 continues by providing to a prescriber 438 by the medication recommender engine 414 the ordered list 434 of recommended medications.

In one or more embodiments, the medication recommender engine 414 is implemented in a neural network and obtains the personal context data 416 and the environmental context data 426 via a web-implemented application programming interface.

Generally, a neural network includes a plurality of computer processors that are configured to work together to implement one or more machine learning algorithms. The implementation may be synchronous or asynchronous. In a neural network, the processors simulate thousands or millions of neurons, which are connected by axons and synapses. Each connection is enforcing, inhibitory, or neutral in its effect on the activation state of connected neural units. Each individual neural unit has a summation function which combines the values of all its inputs together. In some implementations, there is a threshold function or limiting function on at least some connections and/or on at least some neural units, such that the signal must surpass the limit before propagating to other neurons. A neural network can implement supervised, unsupervised, or semi-supervised machine learning. A neural network can be implemented in a stand-alone system or can be implemented in a cloud computing structure.

In one or more embodiments, the personal context data 416 also incorporates information about the given patient's cultural profile.

Based on the ordered list 434 of recommended medications, the method 300 further includes planning logistics and tactics for medication delivery and access.

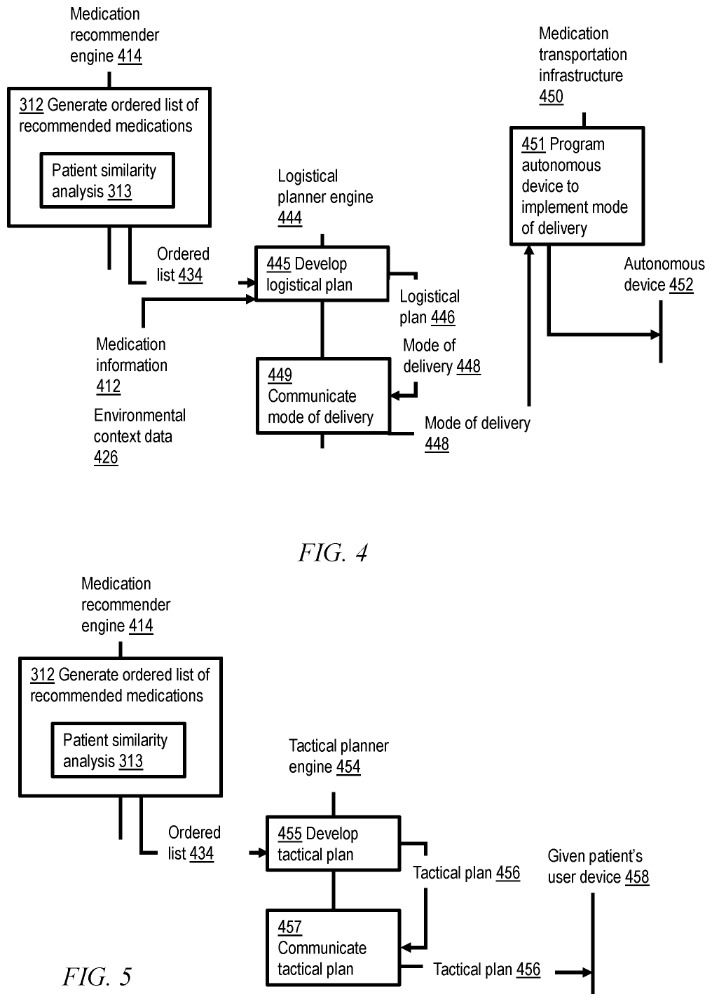

With reference to FIG. 4, a logistical planner engine 444 receives the ordered list 434 from the medication recommender engine 414 and at 445 develops a logistical plan 446 for delivering one of the recommended medications to the given patient via a mode of delivery 448 such as a drone, a courier, etc. The logistical planner engine 444 selects the mode of delivery 448 based on the medication information 412 (e.g., shelf life, storage/transit requirements) and based on the environmental context data 426 (e.g., patient location, weather conditions, available transport modes). At 449 the logistical planner engine 444 then communicates the selected mode of delivery 448 to a medication transportation infrastructure 450. The optimized recommended medication has information on the frequency and duration of the prescription. This is input to the logistical planner engine 444, which is aware of the current environmental context. For a patient with chronic illness, there are usually refills. The logistical planner engine 444 takes all this info and produces a plan of medication delivery. Thus, in response to receiving the selected mode of delivery 448, at 451 the medication transportation infrastructure programs an autonomous device 452 to implement the selected mode of delivery. E.g. in bad weather, bad roads and transport conditions a drone may be programmed to accomplish delivery. Other exemplary autonomous devices include self-driving vehicles and robotic home assistants. A drone (unmanned aerial vehicle) or self-driving road vehicle can be programmed to deliver medication, for example. A robotic home assistant can be programmed to administer medication and/or to meet a drone or self-driving road vehicle to receive medication therefrom, for example.

In another aspect, an alert related to the recommended course of treatment with the medication can be provided to the patient and/or a caregiver. For example, a system implementing aspects of the invention can trigger the alert via SMS (short message service) or other text messaging service.

In any event, for example, a local computing device or a cloud server implementing a system according to one or more embodiments interfaces with a telecommunications network (e.g. via adaptor 20 discussed below) to cause the SMS message to be sent over the network to the patient and/or care giver and/or to program the autonomous device.

One or more embodiments further include delivering the medication with the drone or self-driving road vehicle; receiving the medication with the robotic home assistant; and/or administering the medication with the robotic home assistant, as the case maybe.

Referring to FIG. 5, a tactical planner engine 454 receives the ordered list 434 from the medication recommender engine 414 and at 455 develops a tactical plan 456 for the given patient to access at least a first medication of the ordered list 434. At 457 the tactical planner engine 450 then communicates the tactical plan 456 to the given patient's user device 458 via, e.g., a simple message service (SMS) message. Other modes of communication equally are effective for accomplishing the intent of one or more embodiments of the invention. The tactical planner engine 454 here is used to use the output of the recommendation engine to create a plan over time on how the pharmacy for example should stock. It can also use neural networks to learn the recommendation patterns over different months of the year to predict what to stock. Recommendations can come from many and different care organizations for management of different conditions. The planner combines this for the patient and communicates to her or him. Over a period of time after the patient has been prescribed the recommended medication, the tactical planner engine 454 detects any changes in the patient's context or changes in success stories for a particular regimen. The tactical planner engine 454 on realizing these changes triggers the system to re-run again with the new changes in context; if the recommendation changes then the patient receives an SMS recommending him or her to see his or her doctor.

Given the discussion thus far, and with reference to the several drawing figures, it will be appreciated that, in general terms, an exemplary method, according to an aspect of the invention, includes 302 receiving, by a guideline recommender engine 402, a diagnosis 404 of a medical condition for a given patient; 304 retrieving, by the guideline recommender engine, encoded guidelines 406 for pharmaceutical treatment corresponding to the diagnosis; and in response to the encoded guidelines, 306 generating, by a medication information engine 408 a list 410 of recommended medications with related information 412. The exemplary method further includes 308 modifying the list of recommended medications, by a personalized and contextualized medication recommender engine 414, in response to personal context data 416 for the given patient that is obtained from at least one of a wearable device 418, a smart pill bottle 420, a patient health wallet 422, and an electronic health record 424; 310 further modifying the list of recommended medications by the medication recommender engine in response to environmental context data 426 for the given patient; and 312 generating an ordered list 434 of recommended medications by yet further modifying the list of recommended medications by the medication recommender engine in response to a patient similarity analysis 313 that compares the personal context data of the given patient to personal context data 436 of at least one other patient. The exemplary method also includes 314 providing to a prescriber 438 by the medication recommender engine the ordered list of recommended medications. Furthermore, the exemplary method includes 445 developing a logistical plan 446 including a mode of delivery 448 for a medication selected from the ordered list of recommended medications, and 451 programming an autonomous device 452 to implement the mode of delivery for the selected medication.

In one or more embodiments, the medication recommender engine is implemented in a neural network and obtains the personal context data and the environmental context data via a web-implemented application programming interface.

In one or more embodiments, the patient health wallet is at least one of a system and an application that is owned by the patient and allows the patient to access their health data and get an audit trail of the care providers who see or edit their data.

In one or more embodiments, wherein the patient health wallet has personal information that can be used to define the patient context that is relevant for the medication recommender engine.

In one or more embodiments, the exemplary method also includes obtaining patient preferences by natural language processing on a conversations module data of the patient health wallet.

In one or more embodiments, the smart pill bottle uses sensors for monitoring the status of the pill container and wirelessly transmits this data to a server, wherein the medication recommender engine reads this data to determine a level of adherence to a medication regimen.

In one or more embodiments, the personal context data includes a medication cost factor for each recommended medication.

In one or more embodiments, the personal context data includes an adverse medication reaction factor for each recommended medication.

In one or more embodiments, the environmental context data is obtained from at least one of a weather station 428, an electrical distribution network 430, and a geographical information system 432.

In one or more embodiments, the patient similarity analysis compares at least one of wearable device data, smart pill bottle data, patient health wallet data, and electronic health record data for a given patient and for a group of other patients, and compares outcomes among the group of the other patients to identify a most efficacious medication for those of the group of the other patients who are similar to the given patient.

In one or more embodiments, the patient similarity analysis identifies the most efficacious medication based on the outcomes with shortest time to recovery from an acute condition.

In one or more embodiments, the patient similarity analysis identifies the most efficacious medication based on the outcomes with greatest adherence to medication regimen for a chronic condition.

In one or more embodiments, the patient similarity analysis identifies the most efficacious medication based on the outcomes with slowest progression of a chronic condition.

In another aspect, an exemplary method includes 302 receiving by a guideline recommender engine 402 a diagnosis 404 of a medical condition for a given patient; 304 retrieving by the guideline recommender engine encoded guidelines 406 for pharmaceutical treatment corresponding to the diagnosis; and in response to the encoded guidelines, 306 generating by a medication information engine 408 a list 410 of recommended medications with related information 412. The exemplary method further includes 308 modifying the list of recommended medications by a personalized and contextualized medication recommender engine 414 in response to personal context data 416 for the given patient that includes the given patient's personal preference for allowable side effects of medications.

In one or more embodiments, the exemplary method also includes obtaining the given patient's personal preference for allowable side effects of medications by natural language processing of conversations that are stored in a patient health wallet.

In one or more embodiments, obtaining the given patient's personal preference for allowable side effects of medications includes a smart pill bottle monitoring patient adherence to a medication regimen and transmitting results of such monitoring to a server, and the medication recommender engine reads the results of such monitoring from the server and determines the given patient's level of adherence to the medication regimen.

In one or more embodiments, the exemplary method also includes obtaining the given patient's personal preference for allowable side effects of medications based on the patient's occupation.

In another aspect, an exemplary method includes 302 receiving by a guideline recommender engine 402 a diagnosis 404 of a medical condition for a given patient; 304 retrieving by the guideline recommender engine encoded guidelines 406 for pharmaceutical treatment corresponding to the diagnosis; and in response to the encoded guidelines, 306 generating by a medication information engine 408 a list 410 of recommended medications with related information 412. The exemplary method also includes 308 modifying the list of recommended medications by a personalized and contextualized medication recommender engine 414 in response to personal context data 416 for the given patient that includes the given patient's financial income as well as financial cost for each medication on the list of recommended medications.

In one or more embodiments, the exemplary method also includes 310 further modifying the list of recommended medications by the personalized and contextualized medication recommender engine in response to environmental context data 426 for the given patient that includes availability of each of the list of recommended medications at the given patient's location.

In one or more embodiments, the environmental context data is obtained from at least one of a weather station, an electrical distribution network, and a geographical information system.

In one or more embodiments, the given patient's financial income is estimated based on the patient's occupation.

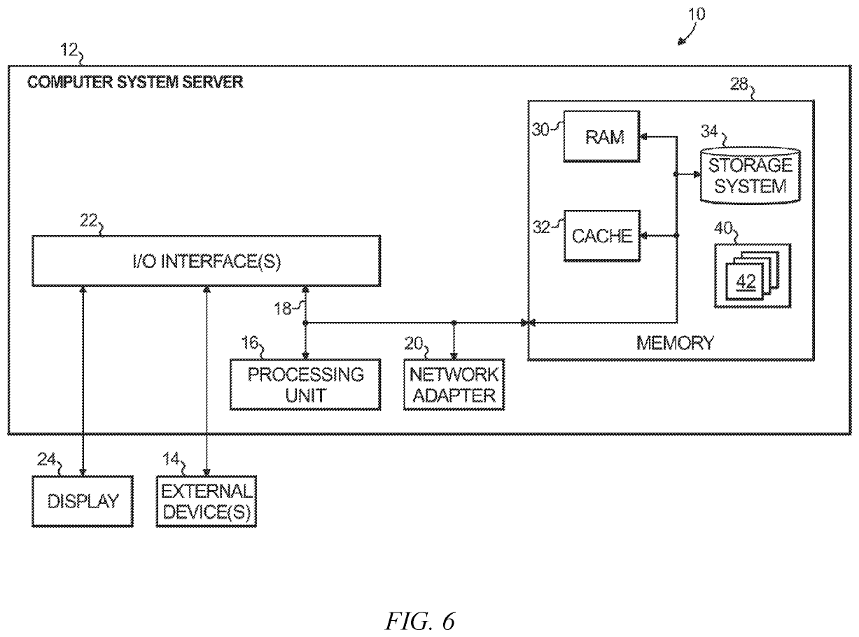

One or more embodiments of the invention, or elements thereof, can be implemented in the form of an apparatus including a memory and at least one processor that is coupled to the memory and operative to perform exemplary method steps, or in the form of a non-transitory computer readable medium embodying computer executable instructions which when executed by a computer cause the computer to perform exemplary method steps. FIG. 6 depicts a computer system that may be useful in implementing one or more aspects and/or elements of the invention, also representative of a cloud computing node according to an embodiment of the present invention.

Referring now to FIG. 6, cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 6, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, and external disk drive arrays, RAID systems, tape drives, and data archival storage systems, etc.

Thus, one or more embodiments can make use of software running on a general purpose computer or workstation. With reference to FIG. 6, such an implementation might employ, for example, a processor 16, a memory 28, and an input/output interface 22 to a display 24 and external device(s) 14 such as a keyboard, a pointing device, or the like. The term “processor” as used herein is intended to include any processing device, such as, for example, one that includes a CPU (central processing unit) and/or other forms of processing circuitry. Further, the term “processor” may refer to more than one individual processor. The term “memory” is intended to include memory associated with a processor or CPU, such as, for example, RAM (random access memory) 30, ROM (read only memory), a fixed memory device (for example, hard drive 34), a removable memory device (for example, diskette), a flash memory and the like. In addition, the phrase “input/output interface” as used herein, is intended to contemplate an interface to, for example, one or more mechanisms for inputting data to the processing unit (for example, mouse), and one or more mechanisms for providing results associated with the processing unit (for example, printer). The processor 16, memory 28, and input/output interface 22 can be interconnected, for example, via bus 18 as part of a data processing unit 12. Suitable interconnections, for example via bus 18, can also be provided to a network interface 20, such as a network card, which can be provided to interface with a computer network, and to a media interface, such as a diskette or CD-ROM drive, which can be provided to interface with suitable media.

Accordingly, computer software including instructions or code for performing the methodologies of the invention, as described herein, may be stored in one or more of the associated memory devices (for example, ROM, fixed or removable memory) and, when ready to be utilized, loaded in part or in whole (for example, into RAM) and implemented by a CPU. Such software could include, but is not limited to, firmware, resident software, microcode, and the like.

A data processing system suitable for storing and/or executing program code will include at least one processor 16 coupled directly or indirectly to memory elements 28 through a system bus 18. The memory elements can include local memory employed during actual implementation of the program code, bulk storage, and cache memories 32 which provide temporary storage of at least some program code in order to reduce the number of times code must be retrieved from bulk storage during implementation.

Input/output or I/O devices (including but not limited to keyboards, displays, pointing devices, and the like) can be coupled to the system either directly or through intervening I/O controllers.

Network adapters 20 may also be coupled to the system to enable the data processing system to become coupled to other data processing systems or remote printers or storage devices through intervening private or public networks. Modems, cable modem and Ethernet cards are just a few of the currently available types of network adapters.

As used herein, including the claims, a “server” includes a physical data processing system (for example, system 12 as shown in FIG. 6) running a server program. It will be understood that such a physical server may or may not include a display and keyboard.

One or more embodiments can be at least partially implemented in the context of a cloud or virtual machine environment, although this is exemplary and non-limiting. Reference is made back to FIGS. 1-2 and accompanying text.

It should be noted that any of the methods described herein can include an additional step of providing a system comprising distinct software modules embodied on a computer readable storage medium; the modules can include, for example, any or all of the appropriate elements depicted in the block diagrams and/or described herein; by way of example and not limitation, any one, some or all of the modules/blocks and or sub-modules/sub-blocks described. The method steps can then be carried out using the distinct software modules and/or sub-modules of the system, as described above, executing on one or more hardware processors such as 16. Further, a computer program product can include a computer-readable storage medium with code adapted to be implemented to carry out one or more method steps described herein, including the provision of the system with the distinct software modules.

One example of user interface that could be employed in some cases is hypertext markup language (HTML) code served out by a server or the like, to a browser of a computing device of a user. The HTML is parsed by the browser on the user's computing device to create a graphical user interface (GUI).

Exemplary System and Article of Manufacture Details

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
receiving by a guideline recommender engine a diagnosis of a medical condition for a given patient;
retrieving by the guideline recommender engine encoded guidelines for pharmaceutical treatment corresponding to the diagnosis;
in response to the encoded guidelines, generating by a medication information engine a list of recommended medications with related information;
modifying the list of recommended medications by a personalized and contextualized medication recommender engine in response to personal context data for the given patient that is obtained from at least one of a wearable device, a smart pill bottle, a patient health wallet, and an electronic health record;
further modifying the list of recommended medications by the medication recommender engine in response to environmental context data for the given patient;
generating an ordered list of recommended medications by yet further modifying the list of recommended medications by the medication recommender engine in response to a patient similarity analysis that compares the personal context data of the given patient to personal context data of at least one other patient;
providing to a prescriber by the medication recommender engine the ordered list of recommended medications;
developing a logistical plan including a mode of delivery for a medication selected from the ordered list of recommended medications; and
programming an autonomous device to implement the mode of delivery for the selected medication,
wherein the medication recommender engine is implemented in a neural network and obtains the personal context data and the environmental context data via a web-implemented application programming interface.

2. The method of claim 1, wherein the patient health wallet comprises at least one of a system and an application that is owned by the patient and allows the patient to access his or her health data and obtain an audit trail of health care providers who see or edit the health data.

3. The method of claim 1, wherein the patient health wallet has personal information that can be used to define the patient context that is relevant for the medication recommender engine.

4. The method of claim 3, further comprising obtaining patient preferences by natural language processing on conversations module data of the patient health wallet.

5. The method of claim 1, wherein the smart pill bottle uses sensors for monitoring the status of the pill container and wirelessly transmits this data to a server, wherein the medication recommender engine reads this data to determine a level of adherence to a medication regimen.

6. The method of claim 1, wherein the personal context data includes a medication cost factor for each recommended medication.

7. The method of claim 1, wherein the personal context data includes an adverse medication reaction factor for each recommended medication.

8. The method of claim 1, wherein the environmental context data is obtained from at least one of a weather station, an electrical distribution network, and a geographical information system.

9. The method of claim 1, wherein the patient similarity analysis compares at least one of wearable device data, smart pill bottle data, patient health wallet data, and electronic health record data for a given patient and for a group of other patients, and compares outcomes among the group of the other patients to identify a most efficacious medication for those of the group of the other patients who are similar to the given patient.

10. The method of claim 9, wherein the patient similarity analysis identifies the most efficacious medication based on the outcomes with shortest time to recovery from an acute condition.

11. The method of claim 9, wherein the patient similarity analysis identifies the most efficacious medication based on the outcomes with greatest adherence to medication regimen for a chronic condition.

12. The method of claim 9, wherein the patient similarity analysis identifies the most efficacious medication based on the outcomes with slowest progression of a chronic condition.

13. A method comprising:
receiving by a guideline recommender engine a diagnosis of a medical condition for a given patient;
retrieving by the guideline recommender engine encoded guidelines for pharmaceutical treatment corresponding to the diagnosis;
in response to the encoded guidelines, generating by a medication information engine a list of recommended medications with related information; and
modifying the list of recommended medications by a personalized and contextualized medication recommender engine in response to personal context data for the given patient that includes the given patient's personal preference for allowable side effects of medications.

14. The method of claim 13, further comprising obtaining the given patient's personal preference for allowable side effects of medications by natural language processing of conversations that are stored in a patient health wallet.

15. The method of claim 13, further comprising obtaining the given patient's personal preference for allowable side effects of medications by a smart pill bottle monitoring patient adherence to a medication regimen and transmitting results of such monitoring to a server, wherein the medication recommender engine reads the results of such monitoring from the server and determines the given patient's level of adherence to the medication regimen.

16. The method of claim 13, further comprising obtaining the given patient's personal preference for allowable side effects of medications based on the patient's occupation.

17. A method comprising:
receiving by a guideline recommender engine a diagnosis of a medical condition for a given patient;
retrieving by the guideline recommender engine encoded guidelines for pharmaceutical treatment corresponding to the diagnosis;
in response to the encoded guidelines, generating by a medication information engine a list of recommended medications with related information;
modifying the list of recommended medications by a personalized and contextualized medication recommender engine in response to personal context data for the given patient that includes the given patient's financial income as well as financial cost for each medication on the list of recommended medications.

18. The method of claim 17, further comprising:
modifying the list of recommended medications by the personalized and contextualized medication recommender engine in response to environmental context data for the given patient that includes availability of each of the list of recommended medications at the given patient's location.

19. The method of claim 18, wherein the environmental context data is obtained from at least one of a weather station, an electrical distribution network, and a geographical information system.

20. The method of claim 17, wherein the given patient's financial income is estimated based on the patient's occupation.
